(19) United States
(12) Patent Application Publication
(10) Pub. No.: US 2021/0260049 A1
(43) Pub. Date: Aug. 26, 2021

(54) Therapeutic Terpene Formulations

(71) Applicant: Boomer Holdings, Inc., Las Vegas, NV (US)

(72) Inventor: Mark Chwajol, Chicago, IL (US)

(73) Assignee: Boomer Holdings, Inc., Las Vegas, NV (US)

(21) Appl. No.: 17/184,353

(22) Filed: Feb. 24, 2021

Related U.S. Application Data
(60) Provisional application No. 62/980,833, filed on Feb. 24, 2020.

(51) Int. Cl. A61K 31/4525; A61K 31/16; A61K 31/015; A61K 31/353; A61K 36/67; A61K 47/14; A61K 47/44; A61K 31/232; A61K 9/00

(52) U.S. Cl. CPC A61K 31/4525; A61K 31/16; A61K 31/015; A61K 31/353; A61K 9/0014; A61K 47/14; A61K 47/44; A61K 31/232; A61K 9/0053; A61K 36/67

(57) ABSTRACT

Therapeutic botanical terpene formulations that promote health and wellness are provided. The formulations employ a synergistic combination of beta-caryophyllene, alpha-pinene, beta-myrcene, cacao extract and black pepper extract that target and activate cannabinoid receptors of the human endocannabinoid system.

### CROSS-REFERENCE TO RELATED APPLICATIONS

The present application claims benefit of U.S. Provisional Patent Application 62/980,833, titled “Therapeutic Terpene Formulations,” filed Feb. 24, 2020, which is incorporated by reference herein in its entirety.

### BACKGROUND

This specification relates generally to therapeutic formulations. More particularly, this specification relates to botanical terpene formulations which promote health and wellness by targeting and activating multiple cannabinoid receptors of the human endocannabinoid system.

Certain exogenous cannabinoids, chemicals found in cannabis, have become ubiquitous due to their wide-ranging therapeutic effects, including anxiety relief and reduction of pain and inflammation. Tetrahydrocannabinol (“THC”) and cannabidiol (“CBD”) are especially popular. However, THC and CBD still face numerous regulatory hurdles. For example, THC is still federally outlawed, and CBD, while legal, cannot be added to foods.

Moreover, while THC and CBD tout numerous health and medical benefits, both may cause adverse side effects. THC, for example, may incur a number of temporary side effects (e.g., slower reaction times and memory loss) due to its psychoactive properties. Furthermore, high THC use may result in negative long-term effects, including a greater risk of psychiatric disorders and cannabinoid hyperemesis syndrome (“CHS”). CBD may cause diarrhea, fatigue, and may also interact with several medications.

There remains a need for legal and generally safe formulations that promote a wide range of the therapeutic benefits, including the ones aforementioned, memory and focus enhancement, improvement of the body's reactivity to stress, promotion of healthy immune function and anti-aging, enhancement of lung and respiratory function, reduction of the appearance of blemishes, improvement of cholesterol, and balancing of blood sugar levels. It would be further beneficial if the ingredients in such formulations worked synergistically with each other when combined.

### SUMMARY

In accordance with the foregoing objectives and others, exemplary botanical terpene formulations are disclosed herein to provide a wide-ranging variety of health benefits. The ingredients employed in the exemplary formulations may work synergistically with one another, such that the beneficial effects of the formulation as a whole are significantly greater than the sum of the effects of each individual ingredient. The disclosed formulations may also, in other embodiments, include or be used in conjunction with cannabinoids such as THC and CBD, and may work synergistically with such cannabinoids in order to emphasize or counteract their effects.

In one embodiment, a composition comprising botanical terpenes is provided. The composition may include comprising active ingredients from about 30% w/w to about 70% w/w of beta-caryophyllene (“BCP”), anandamide (“AEA”), 2-arachidonoylglycerol (“2-AG”), epicatechin, guineensine, and piperine.

The details of one or more embodiments of the subject matter of this specification are set forth in the accompanying drawings and the description below. Other features, aspects, and advantages of the subject matter will become apparent from the description, the drawings, and the claims.

### DETAILED DESCRIPTION

In accordance with the foregoing objectives and others, exemplary therapeutic terpene (i.e., plant-derived dietary ingredients) and terpenoid formulations are disclosed herein that promote a wide range of powerful health and medical benefits. Such benefits include: reduction of pain and inflammation; calming of anxiety and nerves; enhanced memory, alertness, energy, and focus; improvement of mood; improvement of the body's reactivity to stress; promotion of healthy immune function; enhanced lung and respiratory function; reduction of the appearance of blemishes; improvement of cholesterol; balanced blood sugar levels; and aid in the overall process of aging.

The human endocannabinoid system (the “ECS”) helps regulate the functions of sleep, immune response, and pain, and generally assists with the maintenance of homeostasis within the body. When an imbalance is detected, the ECS synthesizes endocannabinoids that interact with (e.g., bind to) the body's cannabinoid receptors and return the body back to a homeostatic state. The four known cannabinoid receptors are: cannabinoid receptor type 1 (“CB1 receptor”), cannabinoid receptor type 2 (“CB2 receptor”), cannabinoid receptor type 3 (“CB3 receptor”), and cannabinoid receptor type 4 (“CB4 receptor”).

CB1 receptors are largely found in the central nervous system (where they regulate a wide variety of brain functions) as well as sporadically throughout the body, including in the skin. CB2 receptors have been found to modulate immune cell functions, both in cellular and in animal models of inflammatory diseases. CB3 receptors are believed to regulate brain and immune function. And CB4 receptors are currently less understood.

The most recognized human endocannabinoids include: AEA, 2-AG, and N-arachidonoyl-dopamine (NADA). These endocannabinoids all bind to CB1, CB2, and CB3 receptors.

Exogenous cannabinoids (e.g., “phytocannabinoids”) are chemicals similar to endocannabinoids and are found in cannabis plants. Phytocannabinoids bind indirectly to the cannabinoid receptors at a different site than endocannabinoids (which bind directly to the cannabinoid receptors), and it has been newly discovered that such interaction modifies (by increasing or decreasing) the ability of the receptors to bind naturally occurring endocannabinoids.

It has been surprisingly found that terpenes—aromatic compounds which create the characteristic scent of many plants—behave similarly to exogenous cannabinoids, by affecting the ECS in an indirect fashion. Exemplary terpenes include, but are not limited to, cannabis, pine, and lavender, as well as fresh orange peel, like phytocannabinoids.

However, terpenes do not include any psychoactive effects present in exogenous cannabinoids such as THC and CBD, and as such, they are able to impart the same benefits of such exogenous cannabinoids with minimal negative effects associated with such exogenous cannabinoids. Terpenes with therapeutic effects and other health benefits include BCP, linalool, and limonene, which among other benefits, elevates mood. Other terpenes and terpenoids with therapeutic effects include, but are not limited to: myrcene, alpha-pinene, humulene, ocimene, terpinolene, pinene, etc.

Furthermore, surprisingly, the terpene formulations have been found to have enhanced beneficial effects on the ECS as a result of the synergy between combinations of the selected terpenes working with other selected terpenes. As such, the beneficial effects of the terpene formulations as a whole are significantly greater than the sum of the effects of each individual terpene alone. The terpene formulations are optimized for not only their beneficial synergistic effects, but for pleasant taste and/or aroma. For example, the formulations may utilize cacao oil/extract, pine tree bark extract, medium chain triglyceride oil (“MCT oil”), etc.

The formulations may, in other embodiments, include or be consumed/applied in conjunction with cannabinoids such as THC and CBD. Such terpene formations have been found to work synergistically with the cannabinoids in order to increase or counteract their effects. For example, BCP combined with alpha-pinene has exhibited enhanced anti-parasitic properties, and myrcene (a terpene occurring in highly fragrant plants, herbs, and fruits) has been shown to accentuate/extend the psychoactive effects of cannabis.

The carefully curated combination of components present in the inventive formulations provide beneficial synergistic effects that promote a wide variety of health benefits with minimal adverse side effects. Indeed, such formations have a significant advantage over currently available therapeutic formulations, which fail to capture the unique spectrum of health-related benefits offered by the inventive formulations.

While certain of the disclosed embodiments are intended for use by humans, other embodiments (discussed in detail below) may be intended for consumption by animals (e.g., dogs, cats, rabbits, reptiles, etc.)

The disclosed therapeutic terpene formulations may be taken internally or externally by a user, and as such, may contain one or more physiologically acceptable excipients. The compositions of the invention include those suitable for oral, inhalation, rectal, ophthalmic (including intravitreal or intracameral), nasal, topical (including buccal and sublingual), vaginal, or parenteral (including subcutaneous, intramuscular, intravenous, intradermal, and intratracheal) administration.

Pharmaceutically acceptable excipients include: coatings, isotonic and absorption delaying agents, binders, adhesives, lubricants, disintegrants, coloring agents, flavoring agents, sweetening agents, absorbants, detergents, carriers, and emulsifying agents.

Generally, the physiologically acceptable carrier(s) is physiologically inert and non-toxic. An example of a physiologically acceptable carrier is purified, preferably sterile, water. Other potential carrier bases to deliver the formulation ingredients orally include: gummies, tablets, drops, pills, sugar pills, glycerine, milk sugar and cane sugar vehicles, alcohol, medicated powders, medicated globules (pellets, pilutes), cones, etc. Preferred embodiments of the terpene formulations may be edible (e.g., in the form of an oil-based tincture, shots, gummies, etc.).

The terms “extract” and “tincture” of an ingredient may generally refer to an essence or concentrate prepared by (1) exposing a part, combination of parts, and/or an entire ingredient to a solvent, (2) separating the solvent from the ingredient, and (3) removing the solvent. In some embodiments, the solvent is alcohol, although in other embodiments, the tinctures may be alcohol-free and instead be oil- and water-based (e.g., with a mix of liquid glycerin and water).

It will be appreciated that in other embodiments, the formulations may be taken nasally, as eye drops, or ear drops. Each of the formulations of the invention can be legally sold as a therapeutic OTC drug per the requirements of the Food and Drug Administration.

In other embodiments, the formulations may be topically applied. Potential carrier bases to deliver the formulation ingredients topically include: gel, alcohol, water/alcohol, cream ointment, salves, lotion, liniment, cream gel, lotion ointment, rub, spray, aerosol, lotion spray, balm rub, gel ointment, lotion cream, poultice, plaster, infusion, decoction, and other methods of preparation. Topical formulations may be rubbed and/or rolled onto the body. Preferred topical embodiments of the terpene formulations that are topical include: massage oils, pain relief roll-on oils, and lotion creams.

Typically, for oral administration of a tincture, the dosage amount is about 1 mL per day. For oral administration of a shot, the dosage amount may be between 37 to 44 mL per day.

The therapeutically effective time window of the formulations may be from about 4 to about 12 hours.

CB5 Compositions

One aspect of the embodiments provides a botanical terpene formulation, which includes a combination of at least five botanically-derived terpenes adapted to regulate the human endocannabinoid system to reach homeostasis (referred to herein as “CB5 compositions”). The CB5 compositions may promote a wide variety of synergistic, therapeutic effects, such as decreasing anxiety, inflammation, depression, insomnia, and/or pain.

In one embodiment, the formulation may be provided in tincture form. Generally, to make the tincture, all oils must be in liquid form, and all ingredients are mixed together. Before use, the tincture should be shaken vigorously in order to ensure that alcohol and oil layers have been adequately reintegrated.

The CB5 compositions may include the active ingredients BCP, alpha-pinene, beta-myrcene, anandamide (“AEA”), 2-Arachidonoylglycerol (“2-AG”), epicatechin, guineensine, and piperine combined with omega-3, -6 fatty acids and antioxidants. The active ingredients (BCP, alpha-pinene, beta-myrcene, AEA, 2-AG, and epicatechin) may generally make up from about 50% to about 70% w/w of the composition. In one embodiment, the active ingredients may be present in amount of about 50% w/w of the composition. In another embodiment, the active ingredients may comprise about 55% w/w of the composition. In another embodiment, the active ingredients may comprise about 60% w/w of the composition. In yet another embodiment, the active ingredients may comprise about 65% w/w of the composition. In yet another embodiment, the active ingredients may comprise about 70% w/w of the composition.

The therapeutic composition may also include inactive ingredients. Such inactive ingredients may comprise from about 30% w/w to about 50% w/w of the composition. The inactive ingredients may include omega-3, -6 fatty acids and antioxidants. The inactive ingredients may also include carriers comprising from about 10% w/w to about 15% w/w of the total composition.

Ingredients of an exemplary CB5 composition are provided in Table 1, below.

TABLE 1
Exemplary CB5 Composition

| Ingredient | % W/W |
|---|---|
| Beta-caryophyllene | About 30-35% |
| Alpha-Pinene | About 25-30% |
| Beta-Myrcene | About 1-5% |
| Anandamide-like compound, 2-Arachidonoylglycerol | About 25-30% |
| (“2-AG”) & Epicatechin (cacao extract) | |
| Guineensine & Piperine | About 1-2% |
| Carrier (omega-3, -6 fatty acids & antioxidants) | At least about |
| | 10% |

As shown, the therapeutic composition includes from about 30% to about 35% w/w BCP. In one particular embodiments, the composition comprises about 31% w/w BCP.

Due to its unique ability to bind with CB2 receptors, BCP has powerful anti-inflammatory, antimicrobial, antibacterial, and antioxidant properties. It has also been found to help relieve anxiety and pain, reduce cholesterol, prevent osteoporosis, and treat seizures. In one embodiment, the BCP may be supplied in the form of black pepper oil and rosemary extract. In other embodiments, the BCP may be additionally or alternatively supplied in the form of clove extract, hops extract, copaiba extract, etc.

The therapeutic composition may comprise from about 25% w/w to about 30% w/w alpha-pinene. In one particular embodiment, the composition comprises about 25% w/w alpha-pinene.

Benefits of alpha-pinene include, but are not limited to, increased alertness, enhanced mood, anti-mutagenic and anti-inflammatory properties, and reduced oil production. Alpha-pinene, taken together with other cannabinoids and/or terpenes, has been found to produce surprising beneficial health effects. When paired with THC, for example, alpha-pinene has been surprisingly found to downplay and counteract the negative side effects of certain compounds such as THC, such as anxiety, paranoia, or short-term memory loss.

Furthermore, the formulation's combination of BCP and alpha-pinene has yielded unexpected enhanced anti-parasitic properties. In one embodiment, the alpha-pinene may be supplied in the form of pine tree bark extract. In other embodiments, the alpha-pinene may be alternatively or additionally supplied by rosemary oil, eucalyptus oil, orange peel oil, etc.

The therapeutic composition may include from about 1% w/w to about 5% w/w beta-myrcene. In one particular embodiment, the composition comprises about 3% w/w beta-myrcene.

Beta-myrcene, in addition to its own anti-inflammatory, sedative, analgesic, and antimutagenic properties, has been also found to surprisingly enhance and amplify the effectiveness of other terpenes by increasing activation of the CB1 receptor and speeding up the entry of certain compounds, like AEA and 2-AG through the blood-brain barrier. Thus, the formulation also includes AEA and 2-AG (discussed below) in order to take advantage of such synergistic effects. In the preferred embodiment, the beta-myrcene may be supplied in the form of rosemary extract. In other embodiments, the beta-myrcene may be alternatively or additionally supplied by lemongrass oil, hop oil, verbena oil, mango oil, thyme oil, bay (Laurus Nobilis) oil, cannabis oil, etc.

The therapeutic composition may include from about 0.5% w/w to about 2% w/w guineensine and piperine (e.g., about 0.5% w/w, about 0.75% w/w, about 1% w/w, about 1.25% w/w, about 1.5% w/w, about 1.75% w/w or about 2% w/w). The general anti-inflammatory effect of guineensine has been surprisingly found to be more potent at this low level of concentration.

The combination of guineensine and piperine have been shown to inhibit cellular endocannabinoid intake, relieve pain, and have anti-inflammatory properties. They have further been surprisingly found to enhance mood boost, particularly when combined with AEA, which is also included in the composition. In the preferred embodiment, the guineensine and piperine may be supplied in the form of black pepper extract. In other embodiments, the guineensine may be alternatively or additionally supplied or supplemented by long pepper extract. In other embodiments, piperine may be alternatively or additionally supplied or supplemented by white pepper extract, long pepper extract, etc.

The therapeutic composition may comprise from about 25% w/w to about 30% w/w 2-Arachidonoylglycerol (“2-AG”), epicatechin, and AEA. In one embodiment, the AEA, 2-AG, and epicatechin may be supplied in the form of cacao extract. In other embodiments, the AEA may be alternatively or additionally supplied or supplemented by galangal extract, maca root, black truffle extract or oil, etc.

The combination of AEA and 2-AG has been found to lower the incidence of seizures. Furthermore, AEA taken together with guineensine, discussed above, (which has been found to be an AEA reuptake inhibitor) and piperine (which has been found to stimulate synthesis and release of AEA) results in the synergistic effect of high levels of AEA resulting in prolonged benefits of the AEA (e.g., enhanced mood).

Furthermore, the combination of AEA, 2-AG, and epicatechin has been shown to regulate feeding behavior, enhance mood, have anti-inflammatory, antimicrobial, antioxidant and neuroprotective properties, manage pain, and treat sleep disorders.

The therapeutic composition also includes from about 10% w/w to about 15% w/w omega-3, -6 fatty acids and antioxidants. In one embodiment, the omega-3, -6 fatty acids and antioxidants may be supplied in the form of MCT oil, which works synergistically with the other components of the composition in order to enhance absorption of the active ingredients and optimize the therapeutic effects of those ingredients. Furthermore, the presence of MCT oil itself has a multitude of benefits, including: lowering cholesterol, lowering blood sugar, improving brain function, burning fat, and reducing hunger.

In one specific embodiment, one serving of the tincture composition may comprise about 17 mg of BCP, about 13.5 mg of anandamide, 2-AG, and epicatechin, about 0.5 mg of guineensine and piperine, and about 6.7 mg of omega-3, -6 fatty acids and antioxidants. Such serving may comprise a volume of about 1 mL.

Alertness Compositions

In one embodiment, a therapeutic composition may be provided that promotes alertness, focus, and energy. Generally, the alertness compositions are optimized for not only beneficial synergistic effects, but also taste and aroma. These alertness compositions may be provided in the form of a tincture.

The alertness compositions may include BCP, cacao extract, and black pepper extract, in substantially similar amounts to those discussed above with respect to the CB5 formulation. The alertness composition may also include at least 10% w/w of a carrier comprising omega-3, -6 fatty acids and antioxidants, such as MCT oil.

The composition may further include from about 15% w/w to about 35% w/w alpha-pinene and d-limonene. D-limonene has been found to possess anti-inflammatory, antioxidant, anti-stress, and disease-preventing properties. In one embodiment, d-limonene may be supplied in the form of orange oil, lemon oil, lime oil, and/or grapefruit oil.

The compositions may further include one or more flavored agents. Such flavoring agents may include, but are not limited to, cinnamon oil, pineapple oil, raspberry oil, orange oil, lemon oil, lime oil, etc.

Relaxation Compositions

In one embodiment, a therapeutic composition may be provided that promotes restfulness and relaxation. Generally, the relaxation compositions may be provided in the form of a tincture.

The relaxation compositions may include BCP, cacao extract, and black pepper extract, in substantially similar amounts to those discussed above with respect to the CB5 formulation. The relaxation compositions may also include at least 10% w/w of a carrier comprising omega-3, -6 fatty acids and antioxidants, such as MCT oil.

The relaxation composition may further include from about 15% w/w to about 35% w/w 3-myrcene, humulene and linalool. Humulene has been shown to exhibit, among other benefits, pain-relieving, anti-cancer, antimicrobial effects. It has been further been unexpectedly shown to enhance weight loss when used in concert with other compounds, such as in those found in cannabis. In one embodiment, humulene may be supplied by one or more of the following including, but not limited to: sage oil, ginseng oil, etc.

Linalool has been shown to exhibit antimicrobial, anti-inflammatory, anticancer, antioxidant, and stress relieving properties. In the preferred embodiments, linalool may be supplied by one or more of the following including, but not limited to: lavender oil, rose oil, basil oil, and neroli oil.

In such embodiments, the compositions may further include a blueberry, cinnamon, or orange flavor and/or aroma, which may be achieved by flavored oils. For example, blueberry oil may be added for a cinnamon flavor, cinnamon oil may be used to achieve a cinnamon flavor.

Various embodiments are described in this specification, with reference to the details discussed above. Numerous specific details are described to provide a thorough understanding of various embodiments. However, in certain instances, well-known or conventional details are not described in order to provide a concise discussion. Therefore, specific structural and functional details disclosed herein are not to be interpreted as limiting, but merely as a basis for the claims and as a representative basis for teaching one skilled in the art to variously employ the embodiments.

The embodiments described and claimed herein and drawings are illustrative and are not to be construed as limiting the embodiments. The subject matter of this specification is not to be limited in scope by the specific examples, as these examples are intended as illustrations of several aspects of the embodiments. Any equivalent examples are intended to be within the scope of the specification. Indeed, various modifications of the disclosed embodiments in addition to those shown and described herein will become apparent to those skilled in the art, and such modifications are also intended to fall within the scope of the appended claims.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

All references including patents, patent applications and publications cited herein are incorporated herein by reference in their entirety and for all purposes to the same extent as if each individual publication or patent or patent application was specifically and individually indicated to be incorporated by reference in its entirety for all purposes.

What is claimed is:

1. A therapeutic composition for modulating the endocannabinoid system (“ECS”) comprising active ingredients from about 30% w/w to about 70% w/w of:
BCP,
anandamide, 2-AG, and epicatechin, and
guineensine and piperine.

2. The therapeutic composition of claim 1, wherein the active ingredients may comprise from about 50% w/w to about 55% w/w of the composition.

3. The therapeutic composition of claim 1, wherein the active ingredients may comprise from about 55% w/w to about 60% w/w of the composition.

4. The therapeutic composition of claim 1, wherein the active ingredients may comprise from about 60% w/w to about 65% w/w of the composition.

5. The therapeutic composition of claim 1, wherein the active ingredients may comprise from about 65% w/w to about 70% w/w of the composition.

6. The therapeutic composition of claim 1, wherein the BCP may comprise from about 30% w/w to about 35% w/w of the composition.

7. The therapeutic composition of claim 1, wherein the guineensine and piperine may comprise from about 0.5% w/w to about 2% w/w of the composition.

8. The therapeutic composition of claim 1, wherein the anandamide, 2-AG, and epicatechin may comprise from about 25% w/w to about 30% w/w of the composition.

9. The therapeutic composition of claim 1, further comprising from about 10% w/w to about 15% w/w of carriers.

10. The therapeutic composition of claim 1, further comprising from about 15% w/w to about 35% w/w of α-pinene and d-limonene, wherein the therapeutic composition enhances energy, alertness, and focus.

11. The therapeutic composition of claim 1, further comprising from about 15% to about 35% of β-myrcene, humulene, and linalool, wherein the therapeutic composition enhances relaxation and restfulness.

12. The therapeutic composition of claim 9, wherein the one or more carriers further comprise MCT oil.

13. The therapeutic composition of claim 9, wherein the one or more carriers further comprise: cinnamon oil, raspberry oil, or blueberry oil.

14. The therapeutic composition of claim 1, wherein the therapeutic composition is formulated for oral, transdermal, topical, or parenteral administration.

15. The therapeutic composition of claim 1, wherein the compositions for oral administration comprise a dosage form selected from the group consisting of: a capsule, a gummy, a powder, and a liquid suspension.

16. The therapeutic composition of claim 1, wherein the compositions for topical administration comprise a dosage form selected from the group consisting of oils and creams.
